(19) United States
(12) Patent Application Publication
(10) Pub. No.: US 2009/0235927 A1
(43) Pub. Date: Sep. 24, 2009

(54) Pacifier for intubated infants

(76) Inventors: Paul Krueger, Tulsa, OK (US); Sean Larsen, Manhattan Beach, CA (US)

Correspondence Address: Lauson & Tarver LLP, 880 Apollo Street, SUITE 301, El Segundo, CA, 90245, US

(21) Appl. No.: 12/077,806

(22) Filed: Mar. 21, 2008

(51) Int. Cl. A61M 16/00; A61J 17/00

(52) U.S. Cl. 128/200.26; 128/200.24; 128/207.14; 606/236; 606/234

(57) ABSTRACT

A pacifier comprising an endotracheal tube holding means for intubated infants. The pacifier further comprises a nipple member attached to a flange member, wherein a channel is disposed longitudinally along the nipple and flange to accommodate an endotracheal tube. A slot, smaller in circumference than the tube, is disposed along the channel and allows the tube to be inserted and removed from the pacifier. The channel is larger in circumference than the tube, so that once the tube is inserted into the pacifier, it is supported in the channel, but can move longitudinally through the channel relative to the pacifier.

### CROSS-REFERENCE TO RELATED APPLICATION

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

Endotracheal tubes prevent infants from using pacifiers properly, and interfere with proper nursing response development. Therefore a pacifier design that allows an infant to nurse while intubated is necessary.

U.S. Pat. No. 5,810,000 to Stevens discloses a pacifier with a slot for accommodating an endotracheal tube. However, this device doesn't allow the tube to move when associated with the pacifier. By contrast, the instant invention allows a user to insert an endotracheal tube into the channel of the pacifier, where it will be securely held, while allowing the pacifier to move laterally along the tube.

U.S. Pat. No. 6,776,157 to Williams et al discloses a medical pacifier for delivering anesthesia and other gases into the oral cavity of a patient. Although this device discloses a channel in the pacifier, the channel is closed, and used only for delivery of gases, including anesthetic gases through the pacifier.

U.S. Pat. No. 6,197,044 to Clayton discloses an improved pacifier configured to facilitate the delivery of fluids, including formula and breast milk. Like Williams, this reference also discloses a closed channel extending through the pacifier.

It is therefore an object of the present invention, to provide a pacifier for intubated infants that holds an endotracheal tube, while encouraging normal nursing development. Another object of the invention is to provide a pacifier that can move along an endotracheal tube, allowing the pacifier to be manipulated and adjusted relative to the tube.

### SUMMARY

The present invention comprises a pacifier for holding an endotracheal tube in intubated infants. The pacifier allows an infant to develop a normal nursing response while intubated. The pacifier comprises a nipple connected to a flange, with a channel extending through the pacifier longitudinally. In order to ensure that an endotracheal tube can move laterally in relation to the pacifier, the channel accommodates an endotracheal tube so that the tube is retained and stabilized by the channel; but can be moved longitudinally relative to the channel. The channel further comprises a slot running along the channel for ingress and egress of the endotracheal tube. The slot is smaller in diameter than the channel, in order to hold the tube in place once the tube is placed in the pacifier.

In order to use the pacifier, an infant is intubated with an endotracheal tube, and the pacifier is brought into contact with the tube, so that the tube crosses through the slot opening, and is disposed in the channel of the pacifier. Thereafter the pacifier can be placed in the mouth of an infant without moving or disturbing the tube. The pacifier can comprise a small size pacifier that holds 1.5 mm, 2.0 mm and 2.5 mm endotracheal tubes, and a larger size that holds 3.0 mm and 3.5 mm endotracheal tubes.

### FIGURES

FIG. 1 is a front perspective view of an endotracheal tube pacifier in accordance with the present invention.

FIG. 2 is a side view of an endotracheal tube pacifier in accordance with the present invention.

FIG. 3 is a front perspective view of an endotracheal tube pacifier in accordance with the present invention with an endotracheal tube installed therein.

FIG. 4 is a rear perspective view of an endotracheal tube pacifier in accordance with the present invention.

### DESCRIPTION

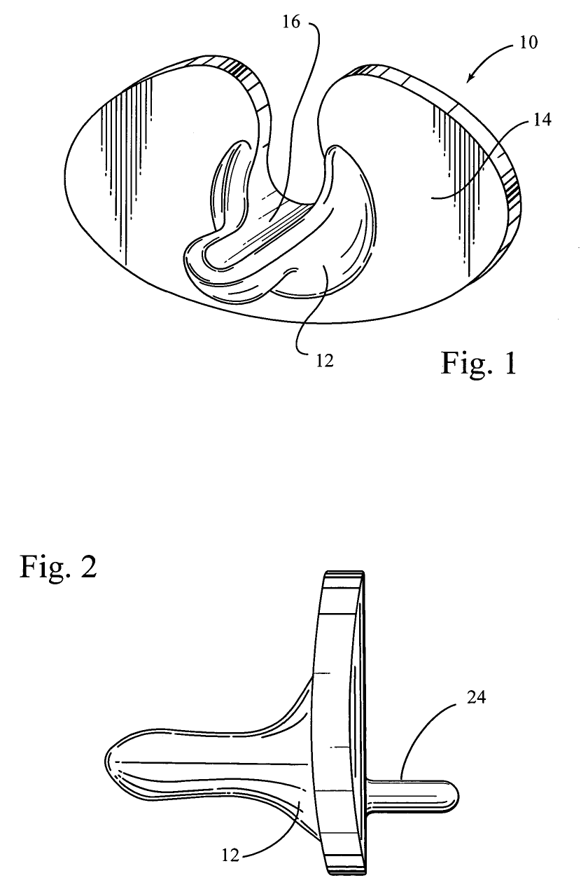

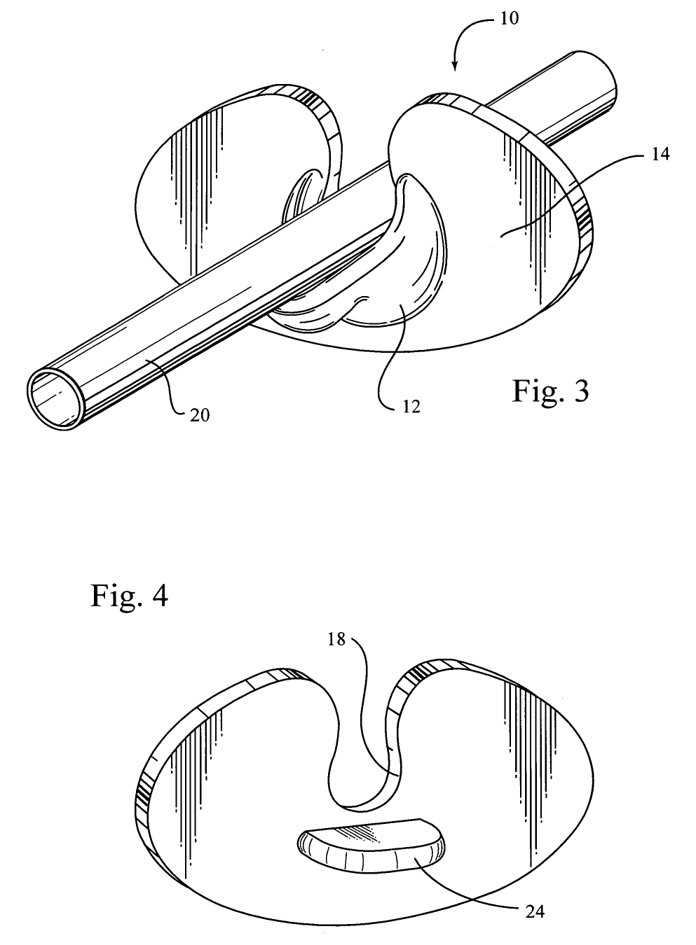

The present invention comprises an improved endotracheal tube accommodating pacifier for intubated infants. The pacifier encourages an infant to develop a normal nursing response while intubated. Referring to FIGS. 1 through 4, the pacifier 10 comprises a nipple 12 with a proximal end connected to a flange 14, and a distal end. The nipple 12 has an increased diameter at the distal end to be placed between the tongue and pallet of an infant.

A channel 16 extends through the nipple 12 and flange 14. The channel 16 accommodates an endotracheal tube 20 in such a manner that the tube 20 is retained and stabilized by the channel 16, but can be moved longitudinally relative to the channel 16. In one preferred embodiment, the channel 16 further comprises a retaining means, including a slot 18 running along the channel. The slot 18 is smaller in diameter than the channel 16, and holds the tube 20 in the channel once the tube 20 is inserted into the slot 18.

In another preferred embodiment, the channel 16 is disposed above the central axis of the nipple, near the edge of the nipple adjacent to the palate of an infant. The pacifier 10 or the nipple 12 may also be comprised of a resilient deformable material that allows the slot 18 to open to accommodate the tube 20. In another embodiment, the pacifier 10, or the flange 14 may be made of a hard material, which causes the tube 20 to deform in order to be inserted into the pacifier 10.

In order to use the pacifier, an infant is intubated with an endotracheal tube. Thereafter, the pacifier is brought into contact with the tube, so that the tube crosses the slot opening, and is disposed in the channel of the pacifier. Thereafter the pacifier can be inserted in the mouth of an infant without moving or disturbing the tube.

It is anticipated that the pacifier will be made in two sizes: A smaller size that holds 1.5 mm, 2.0 mm and 2.5 mm endotracheal tubes, and a larger size that holds 3.0 mm and 3.5 mm endotracheal tubes.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. §112, paragraph 6. In particular, the use of “step of” in the claims herein is not intended to invoke the provisions of 35 U.S.C. §112, paragraph 6.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. An improved endotracheal tube pacifier for intubated infants, comprising;
a. a nipple portion that engages the tongue and palate of an infant,
b. a radially projecting flange portion attached to a distal end of the nipple portion; and
c. a channel extending longitudinally along the length of the nipple portion and through the flange portion that can releasably engage an endotracheal tube through an access slot; and when engaged, permit longitudinal movement of the pacifier along the tube, the circumference of the channel being larger than the circumference of the tube.

2. The pacifier of claim 1, wherein the channel is parallel to the nipple portion, and disposed above the central axis of the nipple.

3. The pacifier of claim 2, wherein the channel is accessible along the top of the nipple.

4. The pacifier of claim 1, wherein the channel comprises a circumference larger than the endotracheal tube associated with the pacifier, and a slot, narrower than the endotracheal tube associated with the pacifier, allowing access to the channel.

5. The pacifier of claim 4, wherein the slot for accessing the channel also extends through the flange portion of the pacifier.

6. The pacifier of claim 1, wherein the pacifier is made from a deformable, resilient material.

7. The pacifier of claim 1, wherein an endotracheal tube is inserted into the pacifier by pressing the tube against the slot with sufficient pressure to cause the slot in the channel to open wide enough to allow the tube into the channel, whereupon the slot will return to its normal width, trapping the tube in the channel.

8. The pacifier of claim 1, wherein the pacifier is able to hold endotracheal tubes with inner diameters selected from the group consisting of 1.5, 2.0 and 2.5 millimeters.

9. The pacifier of claim 1, wherein the pacifier is able to hold endotracheal tubes with inner diameters selected from the group consisting of 3.0 and 3.5 millimeters.

10. The pacifier of claim 1, wherein the nipple portion comprises a proximal extremity and a distal extremity, wherein the distal extremity is connected to the flange, and the proximal extremity comprises a section of enlarged radius that rests against the roof of the mouth of a user and encourages retention of the pacifier.
